(19) United States
(12) Patent Application Publication
(10) Pub. No.: US 2004/0033486 A1
(43) Pub. Date: Feb. 19, 2004

(54) M cell directed vaccines

(76) Inventor: David W. Pascual, Bozeman, MT (US)

Correspondence Address: MORGAN LEWIS & BOCKIUS LLP, 1111 PENNSYLVANIA AVENUE NW, WASHINGTON, DC, 20004, US

(21) Appl. No.: 10/169,492

(22) PCT Filed: Jan. 8, 2001
(86) PCT No.: PCT/US01/00426

(51) Int. Cl. C12Q 1/70; C12Q 1/68; A61K 39/395

(52) U.S. Cl. 435/5; 435/6; 424/130.1; 514/44

(57) ABSTRACT

This invention provides a vaccine that can direct gene transfer to follicle associated epithelium or M cells to induce mucosal immunity using M cell ligands for receptor-mediated endocytosis. Also provided are polynucleotides sequences encoding M cell ligand-polybasic component fusion proteins, host cells, and methods of producing such proteins recombinantly and chemically. Further, methods are described for immunizing animal and human subjects against bacterial, viral, parasitic, fungal infectious agents or cancer and methods for assaying mucosal immunity using this vaccine.

### TECHNICAL FIELD

The present invention is in the general field of vaccine development. The present invention provides methods and compositions useful for, among other purposes, the identification, diagnosis, prevention and treatment of bacterial, viral, parasitic, fungal infectious agents or cancer for human, livestock, and wildlife. More specifically, the present invention provides DNA vaccines directed to follicle-associated epithelium. Even more specifically, the invention is directed to polycation conjugated M cell ligand (e.g., enteric adheins)-DNA complex vaccine compositions and diagnostic and therapeutic uses thereof.

### BACKGROUND OF THE INVENTION

Aspects of this invention are discussed in Wu et al., Gene Therapy (2000) 7(1):61-69, herein incorporated by reference in its entirety. All publications and patent applications mentioned or identified in this specification are incorporated by reference to the same extent as if each individual publication or patent application was specifically and individually indicated to be incorporated by reference.

Recent studies have shown the utility of DNA vaccination for inducing protective immnunity in experimental animals exposed to influenza(Fynan et al., Proc Natl Acad Sci USA (1993) 90:11478-11482 and Robinson et al., Vaccine (1993) 11:957-960), herpes simplex virus (HSV) (Gillichan et al., J Inf Dis (1998) 177:1155-1161), HIV-1 (Boyer), rotavirus (Herrmann et al., J Infec Dis (1996) 174(Suppl.1):S93-S97 and Chen et al., J Virol (1998) 72:5757-5761), and Borrelia burgdorgeri infections (Simon et al., J Immunol (1996) 26:2831-2840). DNA immunization has a number of attractive features including ease of preparation for encoding desired protective immunogens, co-expression of immunogens, co-expression of adjuvant (e.g., cytokines), no requirement for large-scale protein purifications, and ease of delivery. However, conventional DNA vaccine technology immunizes the host at peripheral sites, e.g., intradermal or intramuscular sites. While these methods can elicit systemic cell-mediated and antibody-dependent responses, most infectious agents infect via a mucosal surface, and such DNA immunizations at peripheral sites do not result in optimal mucosal immunity (i.e., both antibody, particularly IgA, and cellular (cytotoxic T lymphocyte (CTL) immunity induction).

This lack of mucosal immunity induction has prompted attempts to deliver DNA vaccines to mucosal surfaces. For example, successful induction of mucosal immunity has been accomplished using DNA vaccines by intraoral jet delivery (Chen et al., Vaccine (1999) 17(23-24):3171-3176); co-administration of a DNA vaccine with a polymer by intranasal injection (Hamajima et al., Clinical Immunol Immunopathol (1998) 88(2):205-210); co-administration of a DNA vaccine with IL-12 (Okada et al., J Immunol (1997) 159(7):3638-3647); intravaginal administration of DNA vaccines (Wang et al., Vaccine (1997) 15(8):821-825); oral delivery of micro-encapsulated DNA vaccines (Jones et al., Dev Biol Stand (1998) 92:149-155); and parenteral and mucosal injection of DNA vaccines (Shroff et al., Vaccine (1999) 18(3-4):222-230). However, these methods generally lack mucosal surface selectivity. Nevertheless, they illustrate the desire to observe mucosal immunity as the end-point in.determining the efficacy of these vaccines.

Transepithelial transport of antigens and pathogens is the first step in the induction of a mucosal immune response. Mucosal inductive tissues are sites in the small intestine or in the nasal passages where vaccine antigens are taken to be processed and presented to mucosal lymphocytes for the development of mucosal immunity (Frey et al., Behring Inst Mitt (1997) 98:376-389). In the intestine, the delivery of antigen across the epithelial barrier to the underlying lymphoid tissue is accomplished by M cells, a specialized epithelial cell type that occurs only in the lymphoid follile-associated epithelium (Frey et al., 1997). Further, such follicle-associated epithelium is found in the nasal lymphoid tissues (believed to be sites of induction of mucosal immune responses to airborne antigens; Giannasca et al., Infect Immun (1997) 65(10):4288-4289). Selective and efficient transport of antigen by M cells is considered an essential requirement for effective mucosal vaccines. Thus, targeting of M cells by taking advantage of their capacity to endocytose particles, including those particles comprising gene transfer vehicles and DNA vaccines, has generated great interest as selective transfer of genes across the follicle-associated epithelium would be advantageous from both investigational and therapeutic standpoints.

Although viruses can be efficient gene transfer vehicles, progress has been made toward developing non-viral delivery systems. Coupling of a specific ligand to vaccines or drugs can be a powerful aid to route compounds to a certain target population. One of the most promising means is by exploiting receptor-mediated endocytosis pathways using selective ligands. In this method, DNA-ligand complexes are internalized by targeted cells when the ligand binds to its respective cell-surface receptor. Such receptor-mediated gene transfer has been accomplished using a variety of receptors by conjugating DNA to their cognate ligands such as asialo-orosomucoid Wu et al., J Biol Chem (1989) 264:16985-16987 and Wu et al., J Biol Chem (1994) 269:11542-11546), transferrin (Lozier et al., Human Gene Ther (1994) 5:313-322; Wagner et al., Proc Natl Acad Sci USA (1990) 87:3410-3414; and Wagner et al., Proc Natl Acad Sci USA (1992) 89:6099-6103), lectins (Batra et al., Gene Ther (1994) 1:255-260), folate (Leamon et al., Biochem J (1993)291:855-860), lung surfactant protein (Ross et al., Human Gene Ther (1995) 6:31-40), insulin (Sobolev et al., J Biol Chem (1998) 273:7928-7933) and would include receptor specific monoclonal antibodies (Chen et al., FEBS Lett (1994) 338:167-169 and Kang et al., J Pharmacol Exp Therapeut (1994) 269:344-350).

Receptor-mediated gene transfer has some advantages over the other methods of in vivo gene transfer. Compared to attenuated viral vectors, it shares tissue-specificity, but receptor-mediated gene transfer minimizes the use of viral gene elements, obviating the concerns regarding genomic integration. Further, it lessens concerns with the proinflammatory properties often associated with viral vectors (Simon et al., Human Gene Ther (1993) 4:771-780; Yang et al., J Virol (1996) 70:7209-7212; and van Ginkel et al., J Immunol (1997) 159:685-693). The DNA-ligand complex is believed to be internalized by receptor-dependent endocytosis rendering transfection to be minimally toxic. The conjugate carrier complex can be designed for cell-specific targeting by selecting the appropriate receptor ligand. For example, efficient transfer of DNA to the intestinal epithelial cells by transferrin-polylysine conjugates and M cell lectins have been used to successfully transfect gastrointestinal cells in vitro (Batra et al., Cancer Gene Ther (1994) 1(3):185-192 and Curiel et al., Am J Respir Cell Mol Biol (1992) 6(3):247-252). However, as transferrin receptors are not restricted to M cells or follicle associated epithelium and as M cell lectins can potentially bind to any α-linked galactose (Giannasca et al., 1997), the use of these systems in vivo is limited.

The surface properties of many enteric pathogens are important in the establishment of the pathogen in the host. For example, enteropathic Escherichia coli (EPEC) induce characteristic attaching and effacing (A/E) lesions on epithelial cells of Peyer's patches (Hartland et al., Mol Microbiol (1999) 32(1):151-158). This event is mediated, in part, by binding of the bacterial outer membrane protein, intimin, to a second EPEC protein, Tir (translocated intimin receptor), which is exported by the bacteria and integrated into the host cell plasma membrane. Both of these protein have been shown to bind to host cells in vitro (Hartland et al, 1999 and DeVinney et al., Cell Mol Life Sci (1999) 55(6-7):961-976).

Reovirus is an enteric pathogen and infects the host following attachment to intestinal Peyer's patch M cells (Lee et al., Virology (1981) 108:156-63 and Mah et al., J Virol (1990) 179:95-103). Thus, as with other enteric pathogens, reovirus exploits M cells as a means to gain entry into the host. Mediating reovirus attachment is the adhesin, σ1, which is expressed as a viral coat protein (Lee et al., 1981). The protein σ1 is a 45 kilodalton protein that polymerizes via its N-terminus (Mah et al., 1990) to form a tetramer when isolated from reovirus-infected cells or purified as a recombinant protein from E. coli (Bassel-Assel-Duby et al., J Virol (1987) 61:1834-1841). In vitro analysis has demonstrated that neutral liposomes comprising σ1 protein can be taken up by rat Peyer's patches Tubas et al., J Microencapsul (1990) 7(3):385-395). Thus, enteric pathogen adhesins make more effective targeting ligands than either transferrin or M cell lectins (Batra et al., 1994, Curiel et al., 1992 and Giannasca et al., 1997).

This invention exploits receptor mediated endocytosis as a means of introducing DNA into cells using M cell ligands for specific targeting of DNA vaccines to follicle associated epithelium of nasal or gastrointestinal origin. We have discovered that, by chemically coupling M cell ligands to a polymeric chain of basic amino acids (e.g., polylysine), DNA can be delivered to appropriate tissue types to obtain enhanced in vivo mucosal IgA antibody and T cell responses against an encoded antigen.

Further, to demonstrate the efficacy of the present vaccine design, we have applied this concept to reporter gene products, β-galactosidase and luciferase, as well as vaccine antigens derived from human immunodeficiency virus (HIV) and Brucella in vivo. Using these systems, enhanced mucosal IgA antibody responses can be demonstrated between animals vaccinated with DNA only (that is, DNA not included in our formulation) and those vaccinated with conjugated DNA complexes.

Our presently formulated DNA vaccine induces improved mucosal IgA antibody responses and promotes sustained CTL responses, demonstrating efficacious vaccination via the mucosa. Further, as the present invention shows the ability of the protein σ1 to mediate efficient gene transfer to the nasal-associated lymphoid tissue (NALT) in vivo, we have demonstrated that systemic and mucosal immunity to intranasally delivered DNA as part of a M cell ligand complex is achievable.

### SUMMARY OF THE INVENTION

The present invention is based, in part, on the observation that a DNA vaccine protected from the mucosal environment can be effectively used to vaccinate a host by targeting the mucosa Data described herein shows that appropriately formulated DNA constructs show improved mucosal IgA antibody responses when compared to DNA applied directly to a mucosal surface. The present invention is further based on the induced anti-vaccine antibody and cellular immune responses produced by vaccinated mice, cattle, and bison. Based on these observations, the present invention provides compositions and methods for use in a variety of animals, particularly humans, livestock, and wildlife.

It is therefore an object of this invention to provide a method for inducing mucosal immunity using receptor mediated endocytosis pathways to deliver nucleotide, particularly DNA, vaccines to specific cells of the follicle associated epithelium, preferably M-cells, for example, of nasal and gastrointestinal origin. It is also an object of this invention to provide DNA vaccine compositions comprising a polypeptide (or other complexing agent) linked electrostatically to (or otherwise associated or complexed with) a DNA structural sequence. Particularly contemplated are polypeptide-DNA complexes, in which the polypeptide is comprised of a polymeric chain of basic amino acid residues and an M cell specific ligand.

The DNA structural sequence preferably encodes an immunogenic antigen from an infectious agent, but also may encode other immunogens, such as a tumor specific antigen, against which the induction of an immune response is desired, but also including antigens against which a host might be tolerized. The present invention provides the ability to produce a previously unknown protein—and to elicit an immune response against such proteins—using the cloned nucleic acid molecules derived, for example, from any given infectious agent be it bacterial, fungal, viral, protozoan, parasitic or protective molecule against cancer.

Consistent with the foregoing, a preferred embodiment of the present invention includes an M cell specific ligand, a nucleic acid sequence encoding an immunogen, and a nucleic acid binding moiety. Preferably, the nucleic acid will be DNA although RNA vaccines are contemplated.

In such vaccines, the binding moiety preferably is a polypeptide, however, other binding and complexing agents may be utilized so long as they stabilize or protect the nucleic acid and protein components of the vaccine from degradtaion and facilitate their delivery, by various routes of administration, to the target mucosal tissues. Thus, for example, a polypeptide binding moiety preferably comprises a polymeric chain of basic amino acid residues and a contemplated polymeric chain would comprise polylysine.

In general, the M cell specific ligand is selected from the group consisting of the protein σ1 of a reovirus, or is (or is derived from) an adhesin of Salmonella or a polio virus. M cell tropic fragments of the foregoing also are contemplated. In a preferred embodiment of the invention, a polypeptide binding moiety would further comprise an M cell specific ligand and may be expressed as a fusion protein.

Also contemplated are nucleotide vaccines in which the immunogen to be delivered to the target mucosal tissue is an immunogen expressed by an infectious agent such as a microorganism or is a tumor specific antigen. Preferred immunogens are derived from or, like an expressed toxin, are associated with a bacterium, protozoan, parasite, virus, fungus, prion, tuberculobacillus, leprosy bacillus, malaria parasite, diphtheria bacillus, tetanus bacillus, Leishmania, Salmonella, Schistoma, measles virus, mumps virus, herpes virus, HIV, cancer and influenza virus. Plasmid vectors in which DNA sequences encode such an immunogen and are operably linked to transcription regulatory elements are a preferred embodiment of the present invention.

The vaccines of the present invention are preferably formulated with a pharmaceutically acceptable excipient or an adjuvant such as an immunomodulator. Examples of contemplated immunomodulators include cytokines, lymphokines, interleukins, interferons and growth factors. Preferably, these vaccines induce a protective immune response in a host vaccinated against the immunogen. In other embodiments of the invention, contemplated vaccines will tolerize a host vaccinated against appropriate immunogens.

Vaccines formulated in unit dosage form, and vaccines packaged with instructions for the use of the vaccine to induce an immune response against said immunogen or disease with which said immunogen is associated are preferred. Therapeutic as well as prophylactic vaccines also are contemplated. Moreover, preferred vaccines are formulated for administration through a route selected from the group consisting of oral, nasal, vaginal, rectal and urethral routes of administration.

Another preferred embodiment of the present invention provides a method for immunizing a host against an immunogen by administering the nucleotide vaccines as described above. In addition, other embodiments of the invention provide a method for assaying for mucosal immunity comprising the steps of administering the vaccine to an animal which is free of infection of the infectious agent whose antigen is to be tested; isolating cells from the animal; and co-incubating said isolated cells with heterologous antigen expressing cells. In this assay, lysing of antigen expressing cells is indicative of mucosal immunity in the vaccinated animal.

Use of the foregoing assay method is contemplated with isolated cells including, for example mucosal B cells, T cells, lamina propria isolates, intraepithelial isolates, Peyer's patches cells, lymph nodes, nasal passages, NALT, adenoids and vaginal epithelium. The the additional step of evaluating the animal's cytokine profile also is contemplated.

A related embodiment of the present invention provides an isolated nucleic acid encoding a fusion protein comprising a nucleic acid binding moiety and an M cell specific ligand. In such nucleic acids, the binding moiety encodes a polymeric chain of basic amino acid residues such as polylysine. Associated vectors comprising these nucleic acids, such as expression vectors, are expressly contemplated. Moreover, the polypeptide expression products of such vectors also may be used as immunogens in vaccines. Contemplated nucleic acids would be in an operable linkage, and would include both sense and antisense orientations relative to transcriptional elements comprising the vector. Host cells comprising or transformed with such vectors are also contemplated.

Another embodiment of the invention includes methods of expressing fusion proteins from such cells. Particularly contemplated are isolated polypeptides comprising a nucleic acid binding moiety and an M cell specific ligand. Optionally, the immunogen also may be encoded by such fusion proteins. It is also contemplated that antibodies may be generated that bind selectively or preferentially to such polypeptides, as opposed to the immunogen or to the M cell specific ligand or nucleic acid binding moiety themselves.

Yet another embodiment of the present invention relates to various kits for assay and other test purposes that include an M cell specific ligand and a nucleic acid binding moiety as well as the other constructs and components described above.

Other objects, features and advantages of the present invention will become apparent from the following detailed description. It should be understood, however, that the detailed description and specific examples, while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows that our recombinant reovirus protein σ1 can bind murine nasal M cells.

FIG. 2 shows sustained mucosal IgA responses against the reporter gene product, luciferase.

FIG. 3 shows induced cytolytic T cell responses against the reporter gene product, β-galactosidase.

FIG. 4 shows the mucosal intestinal IgA response of mice immunized with one of three designated HIV DNA vaccine constructs presenting gp160, gp140(c) or gp 140(s).

FIGS. 5A and 5B show enhanced cytolytic activity (cell-mediated immunity) against target cells expressing HIV gp120 from biopsies from mice immunized intranasally with an M cell-formulated HIV DNA vaccine.

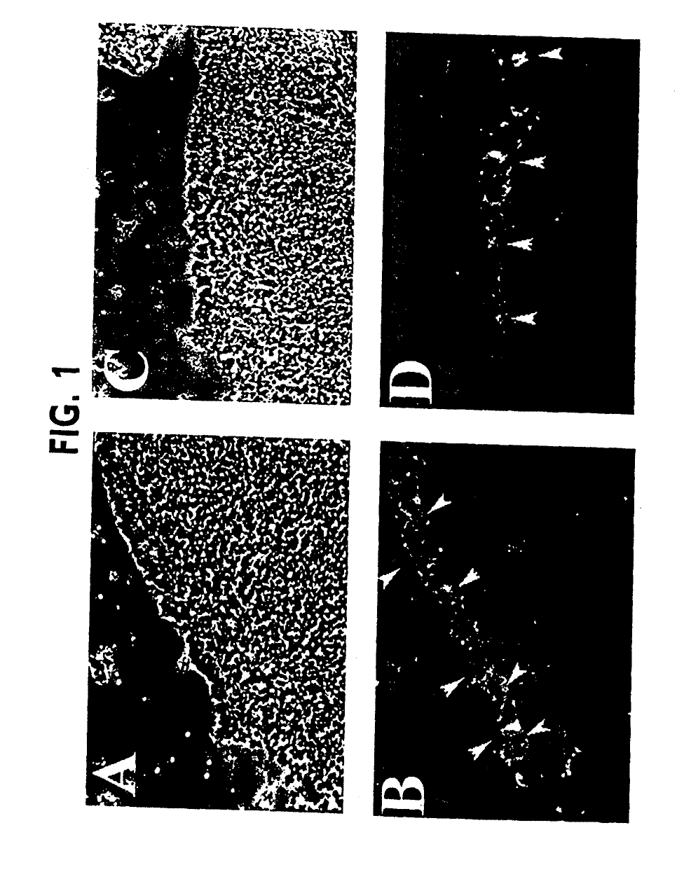

### DETAILED DESCRIPTION OF THE INVENTION

Definitions

As used herein, the term “adjuvant” refers to a substance added to a vaccine to improve the immune response.

As used herein, the term “antibody” refers to an immunoglobulin molecule that has a specific amino acid sequence by virtue of which it interacts only with the antigen that induced its synthesis in cells of the lymphoid series (especially plasma cells) or with antigen closely related to it. Antibodies are classified according to their mode of action as agglutinns, bacteriolysins, haemolysins, opsonins, precipitins, etc.

As used herein, the term “antigen” refers to a substance recognized as foreign by the immune system and can be an immunogen.

As used herein, the term “DNA vaccine” specifically refers to a therapeutic or prophylactic pharmaceutical formulation that contains a nucleic acid that encodes a protein or peptide against which a vaccinated host is induced to mount an immune response, preferably a protective immune response. Preferably, such a DNA vaccine contains a complete eukaryotic expression system encoding the molecular machinery for the expression of such a protein or peptide subunit vaccine. For example, such a DNA vaccine may be encoded in plasmid nucleic acids.

As used herein, the term “enteric adhesin” refers to a peptide, protein, carbohydrate or other class of compound that allows for or facilitates pathogen attachment to M cells as a means to gain entry to the host. For example, a reovirus σ1 protein having a molecular weight of 47 KDa is an enteric adhesin (Nagata et al., Nucleic Acids Res (1984) 12(22):8699-710).

As used herein, the term “expression” refers to the expression of peptides or proteins that are encoded by, for example, the DNA vaccine or associated delivery vector. After expression of such a peptide or protein by, for example, an M cell to which a DNA vaccine has been targeted, such expression by the M cell would lead to the induction of an immune response by a vaccinated host against that encoded immunogen.

As used herein, the term “immunization” refers to a process that increases or enhances an organism's reaction to antigen and therefore improves its ability to resist or overcome infection.

As used herein, the term “immunogen” refers to a antigen that is capable of eliciting (inducing) an immune response. For example, an immunogen usually has a fairly high molecular weight (usually greater than 10,000 daltons). Thus, for example, a variety of macromolecules such as peptides, proteins, lipoproteins, polysaccharides, some nucleic acids, and certain of the teichoic acids, can act as immunogens.

As used herein, the term “infectious agent” refers to a microorganism (or associated substance such as a toxin) that affects or communicates disease through invasion and multiplication of said substance in body tissues, which may be clinically unapparent or result in local cellular injury due to competitive metabolism, toxins, intracellular replication or antigen antibody response.

As used herein, the term “ligand” refers to any molecule that binds to another; in normal usage a soluble molecule such as a hormone or neurotransmitter, that binds to a receptor.

As used herein, the term “M cell(s)” and “follicle associated epithelium” refer to specialized mucosal cells overlying mucosal associated lymphoreticular tissue (MALT), gut associated lymphoid tissue (GALT), bronchus associated lymphoid tissue (BALT) and nasal associated lymphoid tissue (NALT) and any other corresponding mucosal cells that are known to or become known to persons skilled in the art.

As used herein, the term “M cell specific ligand” refers to a molecule that selectively binds to a receptor available on the surface of follicle associated epithelial cell subpopulations, and an M cell specific physiologic effect accompanies that binding (e.g., uptake of pathogen). For example, the enteric adhesin, protein al of reovirus, is an M cell specific ligand. By way of distinction, transferrin and certain other M cell lectins are not considered M cell specific ligands because: 1) the transferrin receptor is not limited to M cells (e.g., neurons express these receptors: Taylor et al., J Comp Physiol (1991) 161(5):521-524) and would not select for follicle associated epithelium subpopulations; and 2) because certain M cell lectins select for α-linked galactose, and many cells possess carbohydrates with said linkages which are not follicle associated epithelium cells (e.g., hepatocytes: Oda et al., J Biol Chem (1988) 263(25):12576-12583). While M cell ligands (rather than M cell specfic ligands) are contemplated for the compositions and methods of certain embodiments of the present invention, the M cell specific ligands are preferred.

As used herein, the term “mucosal” refers to any membrane surface in a host organism, preferably a mammal such as a human being or agriculturally important animal, that is covered by mucous.

As used herein, the term “nucleic acid” includes DNA and RNA molecules and is used synonymously with the terms “nucleic acid sequence” and “polynucleotide.”

As used herein, the term “nucleic acid binding moiety” refers to compositions and substances that are capable of binding to or complexing with DNA and serving as a vehicle to deliver the compositions of the present invention to their target M cells. Polybasic chains of amino acids are particularly contemplated for this purpose, as are, for example, synthetic compounds known to persons skilled in the art that have appropriate ionic charges to form complexes with DNA.

As used herein, “polymeric chain” refers to compounds formed by the joining of smaller, usually repeating, units linked by covalent bonds.

As used herein, the term “polymeric chain of basic amino acids” (i.e., polybasic) refers to a DNA binding sequence that is rich in basic amino acids, such as lysine, arginine, and ornithine, that is typically about ten to 300 residues long. D-isomers of these basic amino acids are suitable so long as the length of the stretch of basic amino acids is within the prescribed length. The polymeric chain of basic amino acids can be a homopolymer of a basic amino acid or it can comprise more than one kind of basic amino acid residue.

As used herein, “polypeptide” refers to an amino acid sequence including, but not limited to, proteins and protein fragments, naturally derived or synthetically produced.

As used herein, the term “reovirus” refers to a genus of the family Reoviridae infecting vertebrates only. Transmission is horizontal and infected species include humans, birds, cattle, monkeys, sheep, swine, and bats. Reovirus 1, reovirus 2, and reovirus 3 infect mammals, and reovirus 1 is the type species.

As used herein, the term “transcriptional factors” refer to a class of proteins that bind to a promoter or to a nearby sequence of DNA to facilitate or prevent transcription initiation.

As used herein, “tumor specific immunogens” refer to immunogens that are preferentially expressed by tumor cells, more preferably immunogens that are selectively expressed by tumor cells.

As used herein, the term “vaccination” refers to the introduction of vaccine into the body of an animal (or host) for the purpose of inducing immunity.

As used herein, the term “vaccine” generally refers to a therapeutic or prophylactic pharmaceutical formulation that contains a component against which a vaccinated host is induced to mount an immune response, preferably a protective immune response. For example, such a component would be a protein encoded by nucleic acids that is expressed by a vaccinated host to form an expressed protein orpeptide subunit vaccine.

General

This invention provides DNA vaccines, preferably polybasic-M cell ligand conjugate-polynucleotide complexes which, when directly introduced into a vertebrate in vivo, including mammals such as humans, induces the expression of encoded proteins within the animal. Prior to the present invention, the art had taught that DNA vaccines represent an efficient method of inducing immunity against a given pathogen if the responsible gene for eliciting protection is identified. As described below, the present inventors have found that the described DNA vaccine formulations improve the targeting of DNA to mucosal inductive tissues. The present invention is based, in part, on the ability of such vaccine formulations to selectively and preferentially target mucosal inductive tissues. Mucosal inductive tissues are sites within the mucosa that support the development of B and T lymphocytes to become stimulated against a specific pathogen or vaccine component or subunit. If the antigen or vaccine can reach this site, there is a strong likelihood that a mucosal immune response will be induced.

To specifically induce such a mucosal immune response, the compositions and methods of the present invention employ ligands formulated to preferentially or specifically target the specialized epithelium that surrounds mucosal inductive tissues referred to as M cells. Thus, a ligand binds M cells to mediate internalization of the dislcosed DNA vaccine. In one embodiment, the M cell ligand is an adhesin of a pathogen, preferably an enteric adhesin of a pathogen, such as a σ1 protein of a reovirus. Additionally, adhesins from Salmonella and poliovirus, as well as other infectious agents having the same tissue tropism would be appropriate. For example, the nucleotide sequences encoding said proteins include but are not limited to polynucleotides comprising nucleotide sequences as set forth in GenBanlc accession numbers: J02325; M10491; AF059719; AF059718; AF059717; AF059716; U74293; and U74292.

In another embodiment, the immunogen may be an enteric adhesin of a pathogen such as an intimin of an enteropathic Escherichia coli. For example, the nucleotide sequences encoding said intimin protein include but are not limited to polynucleotides comprising nucleotide sequences as set forth in GenBank accession numbers: U38618; AJ223063; Y13111; Y13112; AF043226; and U62657. In another embodiment, the immunogen is an enteric adhesin receptor of a pathogen such as an Tir of an enteropathic Escherichia coli. For example, the nucleotide sequences encoding the intimin receptor protein include but are not limited to polynucleotides comprising nucleotide sequences as set forth in accession number: AF113597. In another embodiment, the immunogen is an enteric adhesin of a pathogen such as an invasin of Salmonella typhimurium, Yersinia pestis and pseudotuberculosis and enteropathic Escherichia coli. For example, the nucleotide sequences encoding said invasin proteins include but are not limited to polynucleotides comprising nucleotide sequences as set forth in accession numbers: AF140550; Z48169; X53368; U25631; and M17448.

In general, it is the formulation of an appropriate DNA conjugate or complex (or other delivery vector) to deliver the DNA to a target M cell that improves host immune responses against a specific pathogen or other immunogen. For example, such a vaccine may be comprised of a polybasic conjugate/DNA complex by incorporating an M cell ligand. Thus, for any given immunogen encoded by nucleic acids that can be used for eliciting a host response, such a response can be enhanced through effective targeting mediated by M cell ligands.

In a preferred embodiment, a contemplated polynucleotide is a nucleic acid which contains essential regulatory elements such that upon introduction into a living vertebrate cell, it is able to direct the cellular machinery to produce translation products encoded by the structural gene sequence component of the polynucleotide. In one embodiment of the invention, the polynucleotide is a polydeoxyribonucleic acid comprising immunogen (or antigen) structural genes or fragments thereof operatively linked to a transcriptional promoter(s). In another embodiment of the invention the polynucleotide comprises polyribonucleic acid encoding antigen structural genes or fragments thereof which are amenable to translation by the eukaryotic cellular machinery (ribosomes, tRNAs, and other translation factors). Where the protein encoded by the polynucleotide is one which does not normally occur in that animal except in pathological conditions, (i.e. an heterologous protein) such as proteins associated with human immunodeficiency virus (HIV) and Brucella, the animals' immune system is activated to launch a protective immune response. Because these exogenous proteins are produced by the animals' own tissues, the expressed proteins are processed by the major histocompatibility system (MHC) in a fashion analogous to when an actual infection occurs. The result, as shown in this disclosure, is induction of immune responses against an antigen. Polynucleotides for the purpose of generating immune responses to an encoded protein are referred to herein as polynucleotide or DNA vaccines. The described vaccine works by inducing the vaccinated animal to produce antibodies or cell-mediated immune responses specific for the vaccine. The production of these antibodies or cell-mediated immune responses will protect the host upon subsequent exposure to the infectious agent.

The present formulations encoding various selected antigens may be administered to immunize individuals against, but not limited to, diseases such as tuberculosis (e.g., BCG antigen: Kumar et al., Immunology (1999) 97(3):515-521), leprosy (e.g., antigen 85 complex: Naito et al., Vaccine (1999) 18(9-10):795-798), malaria (e.g., surface antigen MSA-2: Pye et al., Vaccine (1997) 15(9):1017-1023), diptheria (e.g., diptheria toxoid: U.S. Pat. No. 4,691,006), tetanus (e.g., tetanus toxin: Fairweather et al., Infect Immun (1987) 55(11):2541-2545), leishmania (e.g., Leishmania major promastigotes: Lasri et al., Vet Res (1999) 30(5):441-449), salmonella (e.g., covalently bound capsular polysaccharide (Vi) with porin, both isolated from S. typhi.: Singh et al., Microbiol Immunol (1999) 43(6):535-542), schistomiasis (e.g., major antigen of Schistosoma mansoni (Sm28 GST): Auriault et al., Pept Res (1991) 4(1):6-11), measles (e.g., the surface glycoprotein and fusion protein of measles virus: Machamer et al., Infect Immun (1980) 27(3):817-825), mumps (e.g., hemagglutinin-neuraminidase (HN viral gene product: Brown et al., J Infect Dis (1996) 174(3):619-622), herpes (e.g., HSV-2 surface glycoproteins (gB2 and gD2): Corey et al., JAMA (1999) 282(4):331-340), AIDS (e.g., gp160: Pontesilli et al., Lancet (1999) 354(9182):948-949), influenza (e.g., immunodominant peptide from hemagglutinin: Novak et al., J Clin Invest (1999) 104(12):R63-67) and cancer (see Wang R F., J Mol Med (1999) 77(9):640-655). Administration of the formulation to a host results in stimulation of the host's immune system to produce a protective immune response.

The present invention further provides recombinant DNA molecules (rDNAs) that contain a coding sequence. The vaccines are produced using conventional eukaryotic plasmid expression systems for the encoded gene. As used herein, a rDNA molecule is a DNA molecule that has been subjected to molecular manipulation in situ. Methods for generating rDNA molecules are well known in the art, for example, see Sambrook et al., Molecular Cloning (1989). In the preferred rDNA molecules, a coding DNA sequence is operably linked to expression control sequences and/or vector sequences.

The choice of vector and/or expression control sequences to which one of the protein encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a host cell's environment.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories, (Richmond, Calif.), pPL and pKK223 available from Pharmacia, Piscataway, N.J.

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the DNA vaccine molecules of the present invention may further include a selectable marker that is effective in an eukaryotic cell, preferably a drug resistance selection marker. A preferred drug resistance marker is the gene whose expression results in neomycin resistance, ie., the neomycin phosphotransferase (neo) gene. (Southern et al, J Mol Anal. Genet. 1:327-341, 1982.) Alternatively, the selectable marker can be present on a separate plasmid, and the two vectors are introduced by co-transfection of the host cell, and selected by culturing in the appropriate drug for the selectable marker.

The M cell ligand-polybasic conjugates according to the invention may be produced chemically or by the recombinant method. Coupling by the chemical method can be carried out in a manner known per se for the coupling of peptides aid if necessary the individual components may be provided with linker substances before the coupling reaction (this procedure is necessary when there is no functional group suitable for coupling available at the outset, such as a mercapto or alcohol group).

Depending on the desired properties of the conjugates, particularly the desired stability thereof, coupling may be carried out by means of various techniques known to persons skilled in the art, including but not limited to the following techniques. For example, the use of disulphide bridges, which can be cleaved again under reductive conditions (e.g., using succinimidyl pyridyl dithiopropionate, are contemplated. See Jung et al., Biochem Biophys Res Comm 101:599-606 (Jul. 30, 1981). Also contemplated is the use of compounds which are largely stable under biological conditions (e.g., thioethers, by reacting maleimido linkers with sulfhydryl groups of the linker bound to the second component). Further comtemplated is the use of bridges that are unstable under biological conditions, e.g., ester bonds, or using acetal or ketal bonds which are unstable under weakly acidic conditions.

The production of the conjugates according to the invention by the recombinant method offers the advantage of producing precisely defined, uniform compounds, whereas chemical coupling produces conjugate mixtures which then have to be separated.

The recombinant preparation of the conjugates according to the invention can be carried out using methods known for the production of chimeric polypeptides. The present invention further provides methods for producing a protein of the invention using nucleic acid molecules herein described. In general terms, the production of a recombinant form of a protein typically involves the following steps:

First, a nucleic acid molecule is obtained that encodes an M cell ligand protein of the invention. If the encoding sequence is uninterrupted by introns, it is directly suitable for expression in any host. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can; if not normally available, be added to the ends of the coding sequence so as to provide an excisable gene to insert into these vectors. A skilled artisan can readily adapt any host/expression system known in the art for use with the nucleic acid molecules of the invention to produce recombinant protein. The polybasic components may vary in terms of their size and amino acid sequence. Production by genetic engineering has the advantage of allowing the M cell ligand component of the conjugate to be modified, by increasing the ability to bind to the receptor, by suitable mutations, for example, or by shortening the M cell ligand component to the part of the molecule which is responsible for the binding to the receptor. It is particularly expedient for the recombinant preparation of the conjugates according to the invention to use a vector which contains the sequence coding for the M cell ligand component as well as a polyliker into which the required sequence coding for the polybasic component is inserted. In this way, a set of express plasmids can be obtained, of which the plasmid containing the desired sequence can be used as necessary in order to express the conjugate according to the invention.

The nucleic acids which are to be transported into the cell may be DNAs or RNAs, with no restrictions as to the nucleotide sequence. The nucleic acids may be modified, provided that this modification does not affect the polyanionic nature of the nucleic acids; these modifications include, for example, the substitution of the phosphodiester group by phosphorothioates or the use of nucleoside analogues.

With regard to the size of the nucleic acids the invention again permits a wide range of uses. There is no lower limit brought about by the transporting system according to the invention; thus, any lower limit which might arise would be for reasons specific to the particular intended use use or target specificity. It is also possible to convey different nucleic acids into the cell at the same time using the conjugates according to the invention

Within the scope of the present invention it has been possible to demonstrate that M cell ligand-polybasic conjugates can be efficiently absorbed into living cells and internalized. The disclosed conjugates or complexes according to the invention are not apparently harmfull to cell growth. This means that they can be administered repeatedly and thus ensure a constantly high. expression level of the genes and nucleotide sequences inserted into the cell.

The ratio of nucleic acid to conjugate can vary within a wide range, and it is not absolutely necessary to neutralize all the charges of the nucleic acid. This ratio will have to be adjusted for each individual case depending on criteria such as the size and structure of the nucleic acid which is to be transported, the size of the polybasic component and the number and distribution of its charges, so as to achieve a ratio of transportability and biological activity of the nucleic acid which is favorable to the particular application. This ratio can first of all be adjusted coarsely, for example by using the delay in the speed of migration of the DNA in a gel (e.g., using the mobility shift on an agarose gel) or by density gradient centrifugation. Once this provisional ratio has been obtained, it may be expedient to carry out transporting tests with the radioactively labeled complex with respect to the maximum available activity of the nucleic acid in the cell and then reduce the proportion of conjugate if necessary so that the remaining negative charges of the nucleic acid are not an obstacle to transportation into the cell.

The preparation of the M cell ligand-polybasic conjugate/nucleic acid complexes, which are also a subject of the invention, can be carried out using methods known per se for the complexing of polythionic compounds. One possible way of avoiding uncontrolled aggregation or precipitation is to mix the two components together first of all at a high (about 1 molar) concentration of common salt and subsequently to adjust to physiological saline concentration by dialysis or dilution. Preferably, the concentrations of DNA and conjugate used in the complex forming reaction are not too high (more than 100 μg/ml), to ensure that the complexes are not precipitated, as would be known to persons skilled in the art.

A preferred nucleic acid component of the M cell ligand-polybasic moiety-nucleic acid complex according to the invention is an immunogen structural gene encoded by the nucleic acids. The invention further relates to a process for introducing nucleic acid or acids into human or animal cells, preferably forming a complex which is soluble under physiological conditions.

Antibodies against M cell ligand-polybasic moiety protein conjugate or complex may be prepared by immunizing suitable mammalian hosts using the peptides, polypeptides or proteins alone or conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the hapten. The hapten peptides can be extended at either the amino or carboxy terminus with a Cys residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Mistein or modifications which effect immortalization of lymphocytes or spleen cells, as is generally known The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant Fragments of the monoclonals or the polyclonal antisera which contain the immunologically significant portion can be used as antagonists, as well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Regions that bind specifically to the desired regions of the gene products can also be produced in the context of chimeras with multiple species origin.

Attentively, antibodies specific for the M cell ligand polybasic moiety conjugate can be humanized antibodies or human antibodies, as described in U.S. Pat. No. 5,585,089 by Queen et al. See also Riechmann et al., Nature (1988) 332: 323-27.

There are many embodiments of the instant invention which persons skilled in the art can appreciate from the specification. Thus, different transcriptional promoters, terminators, carrier vectors or specific gene sequences may be used successfully. Various methods are known for such constructs which, upon introduction into mammalian cells, induces the expression, in vivo, of the polynucleotide thereby producing the encoded protein. It is readily apparent to those skilled in the art that variations or derivatives of the nucleotide sequence encoding a protein can be produced which alter the amino acid sequence of the encoded protein.

It is well known in the biological arts that certain amino acid substitutions can be made in protein sequences without affecting the function of the protein. Generally, conservative amino acids are tolerated without affecting protein function. Similar amino acids can be those that are similar in size and/or charge properties, for example, aspartate and glutamate, and isoleucine and valine are both pairs of similar amino acids. Similarity between amino acid pairs has been assessed in the art in a number of ways. For example, Dayhoff et al. (1978) in Atlas of Protein Sequence and Structure, Volume 5, Supplement 3, Chapter 22, pages 345-352, which is incorporated by reference herein, provides frequency tables for amino acid substitutions which can be employed as a measure of amino acid similarity. Dayhoff et al.'s frequency tables are based on comparisons of amino acid sequences for proteins having the same function from a variety of different evolutionary sources. The altered expressed protein may have an altered amino acid sequence, yet still elicits immune responses which react with the antigen protein, and are considered functional equivalents. In addition, fragments of the full length genes which encode portions of the fill length immunogenic protein may also be constructed. These fragments should encode a protein or peptide which elicits antibodies that crossreact with the immnunogenic protein, and are considered to be functional equivalents.

The amount of expressible DNA or transcribed RNA to be introduced into a vaccine recipient will have a very broad dosage range and may depend on the strength of the transcriptional and translational promoters used as well as subject size, e.g., human versus bison (i.e., in bison, 5 mg of DNA can be an effective dose). In addition, the magnitude of the immune response may depend on the level of protein expression and on the immunogenicity of the expressed gene product. In general, an effective dose ranges of about 1 ng to 5 mg, 100 ng to 2.5 mg, 1 μg to 750 μg, and preferably about 10 μg to 300 μg of DNA is administered intranasally. It is also contemplated that booster vaccinations may be provided. Following vaccination with M cell ligand-polybasic conjugate-polynucleotide complexes, boosting with the encoded antigen products is also contemplated. Parenteral administration, such as intravenous, intramuscular, subcutaneous or other means of administration of interleukin-12 protein (or other cytokines, e.g. GM-CSF), concurrently with or subsequent to intranasal introduction of the M cell ligand-polybasic conjugate-polynucleotide complex of this invention may be advantageous.

The polynucleotide may be associated with adjuvants or other agents which affect the recipient's immune system. In this case, it is desirable for the formulation to be in a physiologically acceptable solution, such as, but not limited to, sterile saline or sterile buffered saline. The active immunogenic ingredients can be mixed with excipients or carriers which are pharmaceutically acceptable and compatible with the active ingredient. Suitable excipients include but are not limited to water, saline, dextrose, glycerol, ethanol or the like and combinations thereof

In addition, if desired, the DNA vaccine complexes may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmtoyl-sn-glycero-3-hydroxyphosphoryloxy)-ethylamine (CGP 19835A, referred to as MTP-PE); and RIBI, which contains three components extracted from bacteria, monophosphoryl lipid A, trehalose dinycolate and cell wall skeleton (MPL+TDM+CWS) in a 2% squalene/Tween 80 emulsion. The effectiveness of an adjuvant may be determined by measuring the amount of antibodies directed against the immunogen resulting from administration of the immunogen in vaccines which are also comprised of the various adjuvants. Such additional formulations and modes of administration are known in the art and can also be used.

The DNA vaccines of the present invention may be formulated into compositions as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

The M cell ligand-polybasic moiety (or conjugate)-polynucleotide compositions are administered in a manner compatible with the dosage formulation, and in such amount as will be prophylactically and/or therapeutically effective. The quantity to be administered, which is generally in the range of about 100 to 1,000 μg of protein per dose, more generally in the range of about 5 to 500 μg of protein per dose, depends on the subject to be treated, the capacity of the individual's immune system to synthesize antibodies, and the degree of protection desired. Precise amounts of the active ingredient required to be administered may depend on the judgment of the physician and may be peculiar to each individual, but such a determination is within the skill of such a practitioner.

The DNA vaccines of the present invention may be given in a single dose or multiple dose schedule. A multiple dose schedule is one in which a primary course of vaccination may include 1 to 10 or more separate doses, followed by other doses administered at subsequent time intervals as required to maintain and or reinforce the immune response, e.g., at 1 to 4 months for a second dose, and if needed, a subsequent dose(s) after several months.

Immunization by DNA injection allows the ready assembly of multicomponent subunit vaccines. Simultaneous immunization with multiple influenza genes has recently been reported. Donnelly et al., Vaccines (1994) pp 55-59). The inclusion in a DNA vaccine of genes whose products activate different arms of the immune system may also provide thorough protection from subsequent challenge.

The vaccines of the present invention are useful for administration to domesticated or agricultural animals, as well as humans. Vaccines of the present invention may be used to prevent and/or combat infection of any agricultural animals. The techniques for administering these vaccines to animals and humans are known to those skilled in the veterinary and human health fields, respectively.

Except as may be noted hereafter, contemplated techniques for cloning, DNA isolation, amplification and purification, for enzymatic reactions involving DNA ligase, DNA polymerase, restriction endonucleases and the like, and various separation techniques are those well known and commonly employed by those skilled in the art. A number of standard techniques are described in Sambrook et al. (1989) Molecular Cloning: A Laboratory Manual, Second Edition, Cold Spring Harbor Laboratory, Plainview, N.Y.; Maniatis et al. (1982) Molecular Cloning A Laboratory Manual, Cold Spring Harbor Laboratory, Plainview, N.Y.; Wu (ed.) (1993) Meth Enzynol. 218, Part I; Wu (ed.) (1979) Meth Enzymol 68; Wu et al. (eds.) (1983) Meth Enzymol 100 and 101; Grossman et al. (eds.) Meth Enzymol 65; Miller (ed.) (1972) Experiments in Molecular Genetics, Cold Spring Harbor Laboratory, Cold Spring Harbor, N.Y.; Old et al. (1981) Principles of Gene Manipulation, University of California Press, Berkeley, Schleifet al. (1982) Practical Methods in Molecular Biology; Glover (ed.) (1985) DNA Cloning Vol. I and II, IRL Press, Oxford, UK; Hames et al. (eds.) (1985) Nucleic Acid Hybridization, IRL Press, Oxford, UK; Setlow et al. (1979) Genetic Engineering: Principles and Methods, Vols. 1-4, Plenum Press, New York. Abbreviations and nomenclature, where employed, are deemed standard in the field and commonly used in professional journals such as those cited herein.

The following examples are provided to illustrate the present invention without, however, limiting the same thereto.

### EXAMPLES

### Example 1

Production of Recombinant Reovirus σ1 Protein

The cloned protein σ1 cDNA from reovirus serotype 3 strain in the pST3-S1 Banjerjea et al., Virology (1988) 167:601-612) was kindly provided by Dr. Wolfgang K. Jolik from Duke University Medical Center. For its expression in E. coli, using PCR, a 1.4 kb cDNA fragment containing the restriction endonuclease sites, EcoR 1 and Pst 1, was inserted into the polylinker site of an E. coli expression plasmid, pMAL-C2 (New England Biolabs, Beverly, Mass.). The resultant, pMAL-C2-S1, was used to transform E. coli, strain BL21(DE3; Novagen, Madison, Wis.). Upon induction with IPTG, the maltose-binding protein (MBP)::protein σ1 fusion protein was induced in the cytoplasm of E. coli. The clear lysate of E. coli containing the fusion protein was purified by affinity chromatography using amylose resin according to manufacturer's directions (New England Biolabs). This MBP::protein σ1 fusion protein is referred to as recombinant protein σ1.

### Example 2

Preparation of Recombinant Fusion Protein σ1-polylysine-DNA Complex

The recombinant protein σ1 was covalently linked to poly-L-lysine (PL) according to the methods of Wagner et al(1990). Protein σ1 was purified and resuspended in phosphate-buffered saline (PBS), pH 7.3. To generate the dithiopyridine linker, both protein σ1 and PL were each modified with succinimidyl 3-(2-pyridyldithio)propionate (SPDP; Sigma Chemical Co., St. Louis, Mo.). Briefly, in separate vessels, ten milligrams of protein σ1 in 5 ml PBS, pH 7.3, and twenty milligrams of PL (Sigma), with an average chain length of 270 lysine monomers, in 1 ml of 75 mM sodium acetate were each vigorously mixed to react with SPDP in 15 mM ethanolic solution for one hour. The resulting SPDP modified protein σ1 was then dialyzed against PBS, pH 7.3, and the modified PL was then dialyzed against 20 mM sodium acetate to remove unbound SPDP. To generate the mercaptopropionate linker, the resultant PL with dithiopyridine linker was further mixed with 23 mg dithiothreitol (DTT) in sodium bicarbonate solution, pH 7.5, for one hour under argon. The mercaptopropionate PL was dialyzed against 20 mM sodium acetate to remove free DTT. The 10 mg of dithiopyridine-modified protein σ1 was then mixed with the 20 mg of mercaptopropionate-modified PL under argon at room temperature for 18 hours. The resultant reaction generated what is referred to as protein σ1-PL conjugate. This conjugate was dialyzed to remove unreacted mercaptopropionate-PL using a membrane with an exclusion of 100 kilodaltons, against HEPES buffered saline (20 mM HEPES, 100 mM sodium chloride, pH 7.4; HS). Protein σ1-PL concentration was determined using a Bradford assay (Pierce, Rockford, Ill.). For control transfections, MBP-PL conjugates were similarly generated. For the formation of conjugate-DNA complex, the protein σ1-PL conjugate in 125 μl of HS was added dropwise into an equal volume of HS containing the plasmid DNA and incubated at room temperature for 30 minutes to form conjugate-DNA complex.

### Example 3

Cell Ligand Binding Assay

To assess the cell-binding capacity of the protein σ1 and protein σ1-PL conjugates, an immunofluorescent binding assay was performed. The protein σ1 and σ1-PL conjugates were incubated with mouse L cells (CCL-1, American Type Culture Collection, Manassas, Va.), RFL-6 fibroblast cells (CCL-192, ATCC), and Caco-2 cells (HTB-37, ATCC) and binding was assessed using 20 μg/ml of-biotiylated monoclonal anti-reovirus protein σ1 antibody (HB-167, ATCC) and SA-PE (Southern Biotech Assoc., Birmingham, Ala.), and specific binding was then assessed using flow cytometry. No staining was obtained with normal rabbit serum or in the presence of SA-PE only.

### Example 4

Cell Culture and Transfection with Plasmid DNA

The mouse L cells, RFL-6 cells, and Caco-2 cells were used for targeting gene transfer by protein σ1-PL conjugate. The mouse L cells have been used as the in vitro model for reovirus protein σ1 binding studies. Cells were maintained in complete media: Dulbecco's minimum essential medium (DMEM; BioWhittaker, Walkersville, Md.), supplemented with 10% fetal bovine serum (Life Technologies, Grand Island, N.Y.) at 37° C. under 5% CO₂. For Luc assay, 2.5×10⁵ cells were added to each well of the 12-well plate and allowed to adhere overnight. The conjugate-DNA complexes were added and incubated for another 24 hours in complete media. For chloroquine treatment, the cells were incubated with protein σ1-PL-DNA complexes and 100 μM chloroquine for 4 hours at 37° C. Four hours after incubation, the conjugate-DNA complexes were removed, and cells were incubated with complete media for another 24 hours. The cells were lysed to assay reporter gene activity. For β-Gal assay, 5×10⁵ cells were added to each well of 6-well plate and allowed to adhere overnight. The conjugate-DNA complexes containing 8 μg σ1-PL and pCMVβ-gal (Life Technologies), with or without chloroquine, were added and incubated for 24 hours. The cells were then incubated with fresh media for another 24 hours prior to flow cytometry analysis.

### Example 5

Assays for Reporter Gene Detection

The Luc gene was used as a reporter gene to assay protein σ1-PL conjugate-mediated transfection. A 1.4 kb Luc gene fragment flanked with Hind III and EcoR V was extracted from pSPKuci(+) (Promega, Madison, Wis.). The pCMVLuciferase (pCMVLuc) was constructed by ligating the 1.4 kb luciferase gene into the polylinker site in pcDNA3.1(+) (Invitrogen, Carlsbad, Calif.). The cells were lysed with 1× luciferase lysis buffer (Promega, Madison, Wis.). Twenty μl of supernatant of cell lysates were mixed with 100 μl of Luc assay buffer and assayed with a luminometer (LUMAT LB 9507, EG&G Berthold, Germany). The relative light units from the total lysates were used to express the Luc activities produced from each transfection.

Expression of β-Gal was visualized by incubating the transfected cells with PBS solution containing 1 mg/ml of 5-boromo-4-chloro-3-indolyl-β-galactopyranoside (X-Gal, Boeringer Mannheim, Indianapolis, Ind.) at 37° C. for 16 hr. To quantify the transfection efficiency, cells having been transfected with the constricts pCMV-β-Gal (Life Technologies) were harvested, loaded with 200 μM fluorescein-mono-β-D-galactopyranoside (FDG; Molecular Probe, Eugene, Or.) for 30 minutes at 37° C. and diluted with cold PBS to a final concentration of 2.5×10⁵ cells/ml. Flow cytometry analysis was performed using a Becton Dickinson FACSCalibur.

### Example 6

Histochemical Determination of Fusion Protein σ1 Binding to NALT

NALT tissues were collected as previously described (Asanuma et al., J Immunol Methods (1997) 202:123-131 and Heritage et al., Am J Respir Crit Care Med (1997) 156(4 Pt 1): 1256-1262). Palates with visible NALT were washed in DMEM, and prior to binding with biotinylated protein σ1 (following standard procedures), NALT were first incubated in DMEM alone or in the presence of 500 μg/ml of protein σ1 in DMEM with gentle rotation on a GeneMate orbital Shaker (Intermountain Scientific Co., Bountiful, Utah) for 45 minutes at 4° C. NALT were incubated with excess unmodified protein σ1 in order to inhibit biotinylated protein σ1 binding, and thus, show specificity of binding to the NALT. NALT were then washed gently in DMEM and incubated in 50 μg/ml biotinylated protein σ1 in DMEM, and were again rotated gently for 45 min at 4° C. Following incubation, NALT were removed, rinsed gently in PBS, and then arranged in 15 mm by 15 mm Tissue Tek@ Cryomold (Miles Inc., Elkhard, Ind.) with their ventral surfaces oriented toward the bottom of the mold. The palates were then frozen in Tissue Tek® O.C.T. compound embedding media and stored at −80° C. until use. For immunoperoxidase staining, frozen NALT sections, previously treated with biotinylated protein σ1, were cut at 5 mm, air dried, fixed in acetone at 4° C., and air dried before rehydration

Frozen sections were rehydrated in Dulbecco's PBS (DPBS) containing 0.2% normal goat serum (NGS). A 1:250 dilution of SA-HRP conjugate (BioSource International, Camarillo, Calif.) was added for 45 min at room temperature. The location of the HRP was visualized upon reaction with the precipitable substrate, 3-amino-ethylcarbazole (AEC: Sigma).

### Example 7

In vivo Analysis of Intranasal Immunization with σ1 Conjugates Luciferase

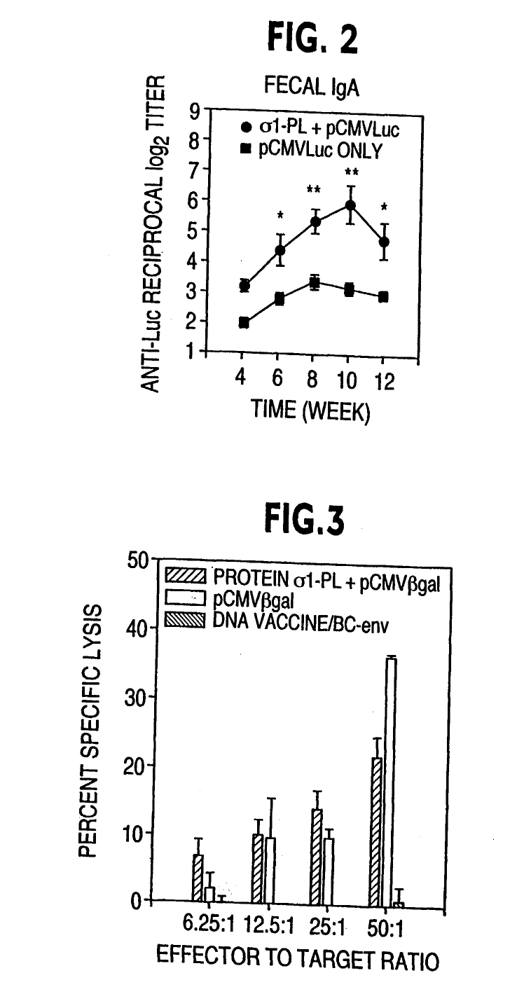

Intranasal (i.n.) immunization with protein σ1-polylysine (PL) conjugate enhances induced mucosal IgA responses in mice. Data depicts the mean endpoint titers (±SE) for mice immunized in with protein σ1-PL-pCMVLuciferase (Luc) or uncomplexed pCMVLuc (5 mice/group). Significant differences between protein σ1-PL-pCMVLuc and pCMVLuc only were determined by student t-test. *p<0.05. **p<0.005. (See FIG. 2).

### Example 8

β-galactosidase

Intranasal (i.n.) immunization with protein σ1-PL-pCMVβ-galactosidase (βgal) stimulates βgal-specific CTL responses in nice. BALB/c mice received three i.n. immunizations with either protein σ1-PL-pCMVβgal or pCMVβgal. Immune splenocytes were able to lyse ⁵¹Cr loaded βgal-expressing fibroblasts (BC-βgal), but not irrelevant BC-envelope (BC-env) targets. The mucosally formulated DNA was as efficient in stimulating βgal-specific CTLs as those mice receiving naked DNA. (See FIG. 3).

### Example 10

HIV

Intranasal (i.n.) immunization with protein σ1-polylysine (PL) conjugate was conducted to enhance induced mucosal IgA responses in mice. The mean endpoint titers (±SE) for mice immunized i.n. with protein σ1-PL-pCMVgp160 and σ1-PL-pCMVgp140 or uncomplexed pCMVgp160 and pCMVgp140 (5 mice/group) was compared. Significant differences between protein σ1-PL-pCMVgp160 and σ1-PL-pCMVgp140 versus pCMVgp160 and pCMVgp140 only were determined by student t-test. Using the mucosal DNA formulation, the same magnitude of IgG antibody response is observed as was observed for the anti-reporter gene responses.

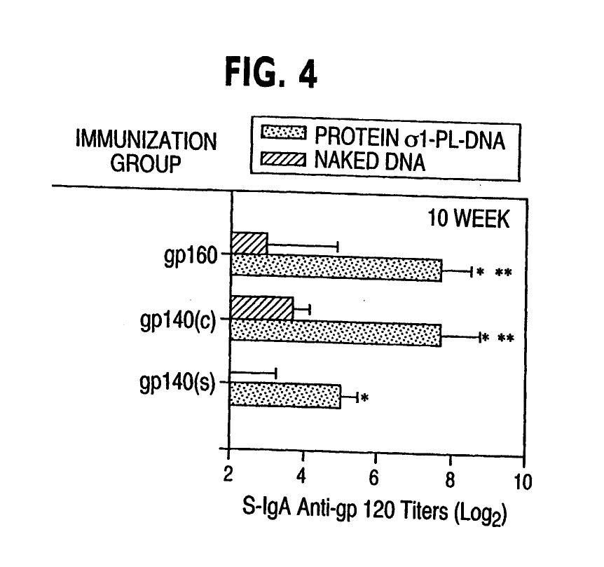

Experimentally, mice were immunized with one of three designated HIV DNA vaccine constructs, that is gp160, gp140(c) and gp 140(s), as indicated in FIG. 4. Each group (5 mice/group) received three intranasal immunizations either of naked DNA or of the identified M cell DNA vaccine formulation. As indicated, the mucosal intestinal IgA response was elevated 10 weeks after the initial immunization when compared to intranasal naked DNA immunization. Thus, the DNA vaccine formulation improved mucosal IgA responses when compared to conventional naked DNA immunization.

### Example 11

Brucella

Intranasal (i.n.) immunization with protein σ1-polylysine (PL) conjugate enhances induced mucosal IgA and IgG responses in bison. The mean endpoint titers (±SE) for bison immunized i.n. with protein σ1-PL-pCMVL7/L12 ribosomal protein or uncomplexed pCMVL7/L12 ribosomal protein (5 bison/group) was compared. Significant differences between protein σ1-PL-pCMVL7/L12 ribosomal protein versus pCMVL7/L12 ribosomal protein only were determined by student t-test. Using our mucosal DNA formulation, we observed increases in serum IgG and vaginal IgA and IgG anti-L7/L12 antibody titer in bison.

### Example 12

HIV gp120

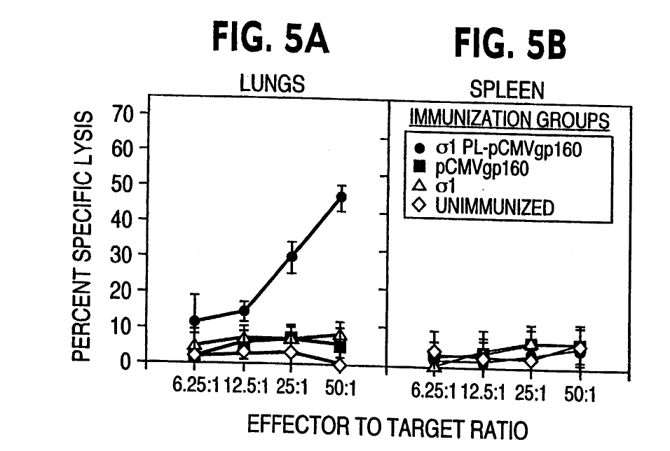

Intranasal immunization with an M cell-formulated HIV DNA vaccine promotes enhanced cytolytic activity (cell-mediated immunity) against target cells expressing HIV gp120 as shown in FIGS. 5A and SB. Mice received a formulated vaccine, naked DNA version, protein sigma1 by the intranasal route three times at one week intervals or were left unimmunized. Mice were sacrificed six weeks subsequent to this initial immunization to procure specified tissues. In a dose-dependent fashion, the lungs from only mice receiving only the formulated vaccine showed effector function. These results show that the vaccine as formulated is superior to naked DNA in stimulating gp120-specific immunity.

Data also indicated that antigen restimulation specifically enhances CTL responses from mice i.n.-immunized with the formulated vaccine as opposed to mice immunized with the naked DNA alone. Pulmonary lymph nodes (LRLN) and splenocytes from immunized mice were restimulated in vitro with cells expressing gp120 or beta-galactosidase (neg. control), and were subsequently examined for cytolytic activity. The observed killing was specific since negative targets were not lysed, and other mechanisms of vaccination failed to stimulate cytolytic activity.

The foregoing detailed description has been given for clearness of understanding only and no unnecessary limitations should be understood therefrom as modifications will be obvious to those skilled in the art. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A composition comprising:
an M cell specific ligand;
a nucleic acid sequence encoding an immunogen; and
a nucleic acid binding moiety.

2. The composition of claim 1, wherein said nucleic acid sequence is a DNA sequence.

3. The composition of claim 1, wherein said binding moiety is a polypeptide.

4. The composition of claim 3, wherein said polypeptide comprises a polymeric chain of basic amino acid residues.

5. The composition of claim 4, wherein said polymeric chain comprises polylysine.

6. The composition of claim 3, wherein said polypeptide further comprises said M cell specific ligand.

7. The composition of claim 1, wherein said immunogen is selected from the group consisting of immunogens expressed by infectious agents and tumor specific antigens.

8. The composition of claim 7, wherein said infectious agent is selected from the group consisting of bacterium, parasite, virus, fungus, prion, tuberculobacillus, leprosy bacillus, malaria parasite, diphtheria bacillus, tetanus bacillus, Leishmania, Salmonella, Schistoma, measles virus, mumps virus, herpes virus, HIV, cancer and influenza virus.

9. The composition of claim 1, wherein said M cell specific ligand is selected from the group consisting of the protein σ1 of a reovirus, adhesin derived from Salmonella and adhesin derived from polio virus and M cell tropic fragments thereof.

10. The composition of claim 9, wherein said M cell specific ligand is the protein σ1 of a reovirus and M cell tropic fragments thereof.

11. The composition of claim 2, wherein said DNA sequence further comprises a plasmid vector in which said DNA sequence encoding an immunogen is operably linked to transcription regulatory elements.

12. A vaccine comprising the composition of any of claims 1 to 11 and a pharmaceutically acceptable excipient.

13. The vaccine of claim 12, which induces a protective immune response in a vaccinated host against said immunogen.

14. The vaccine of claim 12, further comprising an adjuvant.

15. The vaccine of claim 14, wherein said adjuvant comprises an immunomodulator.

16. The vaccine of claim 15, wherein said immunomodulator is selected from the group consisting of cytokines, lymphokines, interleukins, interferons and growth factors.

17. The vaccine of claim 12, wherein the vaccine is formulated in unit dosage form.

18. The vaccine of claim 12, further packaged with instructions for the use of the vaccine to induce an immune response against said immunogen or against the disease with which said immunogen is associated.

19. The vaccine of claim 12, wherein the vaccine is a therapeutic vaccine.

20. The vaccine of claim 12, wherein the vaccine is formulated for administration through a route selected from the group consisting of oral, nasal, vaginal, rectal and urethral routes of administration.

21. A method for immunizing a host against an immunogen, comprising the step of administering the vaccine of claim 12 to the host.

22. A method for assaying for mucosal immunity comprising the steps of
administering the vaccine of claim 12 to an animal which is free of infection of the infectious agent whose antigen is to be tested;
isolating cells from the animal; and
co-incubating said isolated cells with heterologous antigen expressing cells, wherein lysing of antigen expressing cells is indicative of mucosal immunity in the animal.

23. The method of claim 22, wherein said isolated cells are selected from the group consisting of mucosal B cells, T cells, lamina propria isolates, intraepithelial isolates, Peyer's patches cells, lymph nodes, nasal passages, NALT, adenoids and vaginal epithelium.

24. The method of claim 22, comprising the additional step of evaluating the animal's cytokine profile.

25. An isolated nucleic acid encoding a fusion protein comprising a nucleic acid binding moiety and an M cell specific ligand.

26. The nucleic acid of claim 25, wherein said binding moiety comprises a polymeric chain of basic amino acid residues.

27. The nucleic acid of claim 26, wherein said polymeric chain comprises polylysine.

28. The nucleic acid of claim 25, wherein said M cell ligand is selected from the group consisting of: protein σ1 of a reovirus, adhesin derived from Salmonella and adhesin derived from polio virus and M cell tropic fragments thereof.

29. A vector comprising the nucleic acid of any of claims 25 to 28.

30. The vector of claim 29, wherein said vector is an expression vector.

31. A polypeptide comprising the expression product of the vector of claim 30.

32. The vector of claim 29, wherein said nucleic acid is in operable linkage and wherein the operable linkage is selected from the group consisting of sense and antisense orientations relative to transcriptional elements comprising the vector.

33. A host cell comprising the vector of claim 29.

34. A method of expressing a fusion protein comprising the step of expressing the vector of claim 30.

35. An isolated polypeptide comprising a nucleic acid binding moiety and an M cell specific ligand.

36. The polypeptide of claim 35, wherein said binding moiety comprises a polymeric chain of basic amino acid residues.

37. The polypeptide of claim 36, wherein said polymeric chain comprises polylysine.

38. The polypeptide of claim 35, wherein said M cell ligand is selected from the group consisting of: protein σ1 of a reovirus, adhesin derived from Salmonella and adhesin derived from polio virus and M cell tropic fragments thereof.

39. An isolated antibody that binds to the polypeptide of claim 35.

40. A kit comprising:
an M cell specific ligand; and
a nucleic acid binding moiety.

41. The kit of claim 40, wherein said binding moiety is a polypeptide.

42. The kit of claim 41, wherein said polypeptide comprises a polymeric chain of basic amino acid residues.

43. The kit of claim 41, wherein said polymeric chain comprises polylysine.

44. The kit of claim 41, wherein said polypeptide further comprises said M cell specific ligand.

45. The kit of claim 40, wherein said M cell specific ligand is selected from the group consisting of: protein σ1 of a reovirus, adhesin derived from Salmonella and adhesin derived from polio virus and M cell tropic fragments thereof.

46. The kit of claim 45, wherein said M cell specific ligand is the protein σ1 of a reovirus and M cell tropic fragments thereof.
